(19) United States
(12) Patent Application Publication
(10) Pub. No.: US 2024/0000098 A1
(43) Pub. Date: Jan. 4, 2024

(54) SOFT CHEWY CONFECTION COMPRISING HMO

(71) Applicant: DSM IP ASSETS B.V., Heerlen (NL)

(72) Inventors: Zhenbo MA, Kaiseraugst (CH); Conroy Clive SALMON, Kaiseraugst (CH); Paulo Henrique SANTOS, Kaiseraugst (CH)

(21) Appl. No.: 18/251,782

(22) PCT Filed: Oct. 25, 2021
(86) PCT No.: PCT/EP2021/079453

§ 371 Date: May 4, 2023

Related U.S. Application Data
(60) Provisional application No. 63/111,818, filed on Nov. 10, 2020.

(30) Foreign Application Priority Data
Dec. 28, 2020 (CH) 01674/20

(51) Int. Cl. A23G 3/42; A61K 31/702; A23G 3/40

(52) U.S. Cl. CPC A23G 3/42; A61K 31/702; A23G 3/40

(57) ABSTRACT

The present invention relates to a new formulation, which is a soft chewy confection for-mulation, which comprises at least one Human Milk Oligosaccharides (HMOs).

The present invention relates to a new formulation, which is a soft chewy confection for-mulation, which comprises at least one Human Milk Oligosaccharides (HMOs).

Soft chewy confection (or also called soft chew) is a popular form of confectionery and has also been widely used in the field of dietary supplements to deliver many nutrients such as vitamins, carotenoids, minerals, and nutritional lipids.

A soft chew is a solid dosage unit at room temperature that has softer texture and higher moisture/oil content than a conventional tablet or hard chewable tablet. The dosage unit may be designed to be chewed and swallowed by a human or an animal.

Soft chews also increase acceptance of patients who tend to resist swallowing hard tablets or capsules. Unfortunately, soft chews often have high percentage of regular sugar (sucrose) or sugar alcohols in the matrix for good taste and texture.

However, regular sugar has many been known for causing weight gain, increasing risk of diabetes, increasing cellular aging, dental issues etc.; and sugar alcohols may cause gas-trointestinal symptoms. It has been really important to reduce the usage level of sucrose (or sugar alcohols) in confectioneries to produce healthier products for consumers. However, it is often very challenging to reduce the usage level of sucrose in soft chews while still maintaining a good texture. A good texture is an important property for the consumers.

The goal of the present invention to find a way to reduce the regular sugar (also known as white sugar, table sugar or granulated sugar), which is refined sucrose (a disaccharide).

Surprisingly, it was found when at least one HMO in a specific amount (a specific range) is used, then it is possible to reduce the regular sugar significantly without having any negative effect on the properties of the soft chew (such as taste, texture, stability).

Therefore, the present to a soft chewy formulation (SCF) comprising
(i) at least one humectant, and
(ii) at least one wetting agent, and
(iii) at least one (edible) oil, and
(iv) at least one binder, and
(v) at least 0.1-50 weight-% (wt-%), based on the total weight of the soft chewy formulation, of at least one HMO and

which has a water moisture content of less than 8 wt-%, based on the total weight of the soft chewy formulation.

The water moisture content of the soft chewy formulation according to the present invention is preferably less than 6 wt-%, based on the total weight of the soft chewy formulation. Preferably it is in the range of 0.1 to 8 wt-% (more preferably in the range of 0.1 to 6 wt-%), based on the total weight of the soft chewy formulation.

Therefore the present invention relates to a soft chewy formulation (SCF′), which is the soft chewy formulation (SCF) having a water moisture content of less than 6 wt-%, based on the total weight of the soft chewy formulation.

Therefore the present invention relates to a soft chewy formulation (SCF″), which is the soft chewy formulation (SCF) having a water moisture content of 0.1 to 8 wt-%, based on the total weight of the soft chewy formulation.

Therefore the present invention relates to a soft chewy formulation (SCF′″), which is the soft chewy formulation (SCF) having a water moisture content of 0.1 to 6 wt-%, based on the total weight of the soft chewy formulation.

The soft chewy formulation according to the present invention has a nice mouth feeling and a usual hardness.

A typical hardness (measured by a textual analyser) is in a range of 1.2 kg to 4.0 kg.

Therefore the present invention relates to a soft chewy formulation (SCF1), which is the soft chewy formulation (SCF), (SCF′), (SCF″) or (SCF″) having a hardness (measured by a textual analyser) in the range of 1.2 kg to 4.0 kg.

Due to the addition of the HMO (or mixture of HMO), the amount of sugar or sugar alcohols in the soft chewy formulation can be reduced significantly.

Human milk oligosaccharides (HMOs) are a family of structurally diverse unconjugated glycans that are highly abundant in and unique to human milk. Originally, HMOs were proposed to be prebiotic “bifidus factors,” or human milk glycans found to promote growth in Bifidobacterial species of the gut and found uniquely in the stool of breast fed infants compared to formula fed infants.

HMOs are composed of the five monosaccharides glucose (Glc), galactose (Gal), N-acetylglucosamine (GlcNAc), fucose (Fuc) and sialic acid (Sia), with N-acetylneuraminic acid (Neu5Ac) as the predominant if not only form of Sia. More than two hundred different HMOs have been identified so far. The most important ones are 2′-fucosyllactose (2′ FL), lacto-N-neotetraose (LNnT), 3-fucosyllactose (3FL), difucosyllactose (DFL), Lacto-N-fucopentaose I (LNFP I), 3″Sialyllactose Sodium Salt (3′SL), 6″Sialyllactose Sodium Salt (6′SL), and Lacto-N-Tetraose (LNT).

In the context of the present invention the most important ones are 2′-fucosyllactose (2′ FL) and lacto-N-neotetraose (LNnT).

HMOs can be isolated from breast milk or they can be produced chemically or biochemi-cally. HMOs are available commercially from a variety of producers.

For the purpose of the present invention the source of the HMO is not essential. It is clear that HMOs from different sources can be used.

Several studies have reported the health benefits of HMOs, which include modulation of the intestinal microbiota, anti-adhesive effect against pathogens, modulation of the intestinal epithelial cell response, and development of the immune system.

Therefore, HMOs have very positive effect when consumed by humans and/or animals. In the embodiment of the present invention the HMO does not only serve as active ingre-dient with a health benefit for humans and animals, but it has also as stated above a positive effect on the property of the soft chew.

Therefore the present invention relates to a soft chewy formulation (SCF2), which is the soft chewy formulation (SCF), (SCF′), (SCF″), (SCF″) or (SCF1), wherein the HMO are chosen from the group consisting of 2′-fucosyllactose (2′ FL), lacto-N-neotetraose (LNnT), 3-fucosyllactose (3FL), difucosyllactose (DFL), Lacto-N-fucopentaose I (LNFP I), 3″Sialyllactose Sodium Salt (3′SL), 6″Sialyllactose Sodium Salt (6′SL), and Lacto-N-Tetraose (LNT).

Therefore the present invention relates to a soft chewy formulation (SCF2′), which is the soft chewy formulation (SCF), (SCF′), (SCF″), (SCF″) or (SCF1), wherein the HMO are chosen from the group consisting of 2′-fucosyllactose (2′ FL) and lacto-N-neotetraose (LNnT).

The amount of the at least one HMO is 0.1-50 wt-%, based on the total weight of the soft chewy formulation (Preferably 5-50 wt-%, more preferably 5-45 wt-%, especially preferred 10-40 wt-%, based on the total weight of the soft chewy formulation).

Therefore the present invention relates to a soft chewy formulation (SCF3), which is the soft chewy formulation (SCF), (SCF′), (SCF″), (SCF′″), (SCF1), (SCF2) or (SCF2′), wherein the content of the at least one HMO is 5-45 wt-%, based on the total weight of the soft chewy formulation.

Therefore the present invention relates to a soft chewy formulation (SCF3″), which is the soft chewy formulation (SCF), (SCF′), (SCF″), (SCF′″), (SCF1), (SCF2) or (SCF2′), wherein the content of the at least one HMO is 10-40 wt-%, based on the total weight of the soft chewy formulation.

The soft chewy formulation according to the present invention also comprises at least humectant.

Usually the at least one humectant is used in an amount of 0.5 to 8 wt-%, based on the total weight of the chewy formulation. Preferably 0.5 to 7 wt-%, more preferably 0.5 to 5 wt-%, based on the total weight of the chewy formulation.

Therefore the present invention relates to a soft chewy formulation (SCF4), which is the soft chewy formulation (SCF), (SCF′), (SCF″), (SCF′″), (SCF1), (SCF2), (SCF2′), (SCF3), (SCF3′) or (SCF3″), wherein the content of the at least one humectant is 0.5 to 8 wt-%, based on the total weight of the chewy formulation.

Therefore the present invention relates to a soft chewy formulation (SCF4′), which is the soft chewy formulation (SCF), (SCF′), (SCF″), (SCF′″), (SCF1), (SCF2), (SCF2′), (SCF3), (SCF3′) or (SCF3″), wherein the content of the at least one humectant is 0.5 to 7 wt-%, based on the total weight of the chewy formulation.

Therefore the present invention relates to a soft chewy formulation (SCF4″), which is the soft chewy formulation (SCF), (SCF′), (SCF″), (SCF′″), (SCF1), (SCF2), (SCF2′), (SCF3), (SCF3′) or (SCF3″), wherein the content of the at least one humectant is 0.5 to 5 wt-%, based on the total weight of the chewy formulation.

The humectants used in the soft chewy formulation according to the present invention are the commonly used ones. Suitable humectants are non-ionic polyols like glycerin or glycerol and its triester (triacetin), which are used for the purpose of controlling viscosity and texture.

Therefore the present invention relates to a soft chewy formulation (SCF5), which is the soft chewy formulation (SCF), (SCF′), (SCF″), (SCF′″), (SCF′), (SCF″), (SCF′″), (SCF1), (SCF2), (SCF2′), (SCF3), (SCF3′), (SCF3″), (SCF4), (SCF4′) or (SCF4″), wherein the at least one humectant is a non-ionic polyol.

Therefore the present invention relates to a soft chewy formulation (SCF5′), which is the soft chewy formulation (SCF5), wherein the at least one humectant is chosen from the group consisting of glycerin or glycerol and as well as its triester.

The soft chewy formulation according to the present invention also comprise at least one wetting agent. Usually the amount of such a wetting agent (or mixture of wetting agents) is in the range of 0.05 to 4.0 wt-%, based on the total weight of the soft chewy formulation. (preferably 0.1 to 2.0 wt-%, more preferably 0.5 to 1.0 wt-%, based on the total weight of the soft chewy formulation).

Therefore the present invention relates to a soft chewy formulation (SCF6), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5) or (SCF5′), wherein the content of the at least one wetting agent is in the range of 0.05 to 4.0 wt-%, based on the total weight of the soft chewy formulation.

Therefore the present invention relates to a soft chewy formulation (SCF6′), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5) or (SCF5′), wherein the content of the at least one wetting agent is in the range of 0.1 to 2.0 wt-%, based on the total weight of the soft chewy formulation.

Therefore the present invention relates to a soft chewy formulation (SCF6″), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5) or (SCF5′), wherein the content of the at least one wetting agent is in the range of 0.5 to 1.0 wt-%, based on the total weight of the soft chewy formulation.

Any commonly used (food grade) wetting agent (or emulsifiers) may be used in the soft chewy formulation according to the present invention. Such agents are used to maintain texture, quality and freshness of the soft chewy formulation,

Common food emulsifiers used are lecithin and lecithin derivatives, monoglyceride derivatives (such as ethoxylated monoglycerides, acetoglycerides and diacetyl tartaric esters of monoglycerides), fatty acid derivatives (such as polyglycerol esters (PGE), propylene glycol esters (PGMS), stearoyl lactylates, sucrose esters, sorbitan esters, polysorbates and sucrose esters), polysorbate 80 (which is a derivative of polyethoxylated sorbitan and oleic acid), and sorbitan monostearate.

Therefore the present invention relates to a soft chewy formulation (SCF7), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′) or (SCF6″), wherein the at least one wetting agent is chosen from the group consisting of lecithin (and lecithin derivatives), monoglyceride derivatives (such as ethoxylated monoglycerides, acetoglycerides and diacetyl tartaric esters of monoglycerides), fatty acid derivatives (such as polyglycerol esters (PGE), propylene glycol esters (PGMS), stearoyl lactylates, sucrose esters, sorbitan esters, polysorbates and sucrose esters), polysorbate 80 (which is a derivative of polyethoxylated sorbitan and oleic acid), and sorbitan monostearate

The soft chewy formulation according to the present invention also comprises at least one oil, which is an edible oil.

Usually the at least one oil is used in an amount of 1 to 10 wt-%, based on the total weight of the chewy formulation. Preferably 2 to 8 wt-%, more preferably 3 to 7 wt-%, based on the total weight of the chewy formulation.

Therefore the present invention relates to a soft chewy formulation (SCF8), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″) or (SCF7), wherein the content of the at least one oil is 1 to 10 wt-%, based on the total weight of the chewy formulation.

Therefore the present invention relates to a soft chewy formulation (SCF8′), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″) or (SCF7), wherein the content of the at least one oil is 2 to 8 wt-%, based on the total weight of the chewy formulation.

Therefore the present invention relates to a soft chewy formulation (SCF8″), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″) or (SCF7), wherein the content of the at least one oil is 3 to 7 wt-%, based on the total weight of the chewy formulation.

Any usually used edible oil can be used in the context of the present invention.

Suitable edible oils are for example canola oil, soybean oil, sunflower oil, coconut oil, hemp oil, sunflower oil, soybean oil, pumpkin oil, cottonseed oil, corn oil, palm oil, castor bean oil, sesame oil, peanut oil, rapeseed oil and safflower oil.

Therefore the present invention relates to a soft chewy formulation (SCF9), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′) or (SCF8″), wherein the at least one edible oil is chosen from the group consisting of canola oil, soybean oil, sunflower oil, coconut oil, hemp oil, sunflower oil, soybean oil, pumpkin oil, cottonseed oil, corn oil, palm oil, castor bean oil, sesame oil, peanut oil, rapeseed oil and safflower oil.

The soft chewy formulation according to the present invention comprises at least one binder.

Usually the at least one binder is used in an amount of 10 to 45 wt-%, based on the total weight of the chewy formulation. Preferably 15 to 40 wt-%, more preferably 20 to 35 wt-%, based on the total weight of the chewy formulation.

Therefore the present invention relates to a soft chewy formulation (SCF10), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″) or (SCF9), wherein the content of the at least one binder is 10 to 45 wt-%, based on the total weight of the chewy formulation.

Therefore the present invention relates to a soft chewy formulation (SCF10′), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″) or (SCF9), wherein the content of the at least one binder is 15 to 40 wt-%, based on the total weight of the chewy formulation.

Therefore the present invention relates to a soft chewy formulation (SCF10″), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″) or (SCF9), wherein the content of the at least one binder is 20 to 35 wt-%, based on the total weight of the chewy formulation.

Any usually used (food grade) binder can be used in the context of the present invention. They may natural as well as synthetic ones.

Suitable binders are for example sucrose, sorbitol, mannitol, xylitol, liquid glucose, acacia, tragacanth, gelatin, starch, pregelatinized starch, alginic acid, cellulose, methyl cellulose, ethyl cellulose, hydroxy propyl methyl cellulose (HPMC), hydroxy propyl cellulose, sodium carboxy methyl cellulose, polyvinyl pyrrolidone (PVP), polyethylene glycol (PEG), polyvinyl alcohols and polymethacrylates.

Therefore the present invention relates to a soft chewy formulation (SCF11), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″), (SCF9), (SCF10), (SCF10′) or (SCF10″), wherein the at least one binder is chosen from the group consisting of sucrose, sorbitol, mannitol, xylitol, liquid glucose, acacia, tragacanth, gelatin, starch, pregelatinized starch, alginic acid, cellulose, methyl cellulose, ethyl cellulose, hydroxy propyl methyl cellulose (HPMC), hydroxy propyl cellulose, sodium carboxy methyl cellulose, polyvinyl pyrrolidone (PVP), polyethylene glycol (PEG), polyvinyl alcohols and polymethacrylates.

The soft chews according to the present invention include further ingredients, which are usually used to produce soft chews, such as bulking agents, sweeteners, nutritional lipids, flavoring agents, disintegrating agents, preservatives, lubricating agents, and anti-sticking agents. Such ingredients are useful for enhancing the manufacturability, texture and ap-pearance of the product. Those of ordinary skill in the art will be familiar with such inactive ingredients, which need not include water for use in the invention.

Such additional ingredients may be present in the amount of up to 45 wt-%, based on the total weight of the soft chewy formulation.

Therefore the present invention relates to a soft chewy formulation (SCF12), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″), (SCF9), (SCF10), (SCF10′), (SCF10″) or (SCF11), wherein the soft chews comprise further ingredients chosen from the group consisting of bulking agents, sweeteners, nutritional lipids, flavoring agents, disintegrating agents, preservatives, lubricating agents, and anti-sticking agents.

Therefore the present invention relates to a soft chewy formulation (SCF12′), which is the soft chewy formulation (SCF12), wherein the content of the further ingredients are up to 45 wt-%, based on the total weight of the soft chewy formulation.

As stated above the advantage of the present invention is that the amount of regular sugar (powdered sugar) is reduced.

The amount of regular sugar is less than 40 wt-%, preferably less than 35 wt-%, more preferably less than 30 wt-%, especially preferably than 20 wt-%, more especially preferably less than 15 wt-%, based on the total weight of the soft chewy formulation. Usually, the content of regular sugar is 8 to 45 wt-%, preferably 8-35 wt-%, more preferably, 10-30 wt-%, especially preferred 8-15% wt-%, based on the total weight of the soft chewy formulation.

Therefore the present invention relates to a soft chewy formulation (SCF13), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″), (SCF9), (SCF10), (SCF10′), (SCF10″), (SCF11), (SCF12) or (SCF12), wherein the content of regular sugar is less than 40 wt-%, based on the total weight of the soft chewy formulation.

Therefore the present invention relates to a soft chewy formulation (SCF13′), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″), (SCF9), (SCF10), (SCF10′), (SCF10″), (SCF11), (SCF12) or (SCF12), wherein the content of regular sugar is less than 35 wt-%, based on the total weight of the soft chewy formulation.

Therefore the present invention relates to a soft chewy formulation (SCF13″), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″), (SCF9), (SCF10), (SCF10′), (SCF10″), (SCF11), (SCF12) or (SCF12), wherein the content of regular sugar is less than 30 wt-%, based on the total weight of the soft chewy formulation.

Therefore the present invention relates to a soft chewy formulation (SCF13′″), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″), (SCF9), (SCF10), (SCF10′), (SCF10″), (SCF11), (SCF12) or (SCF12), wherein the content of regular sugar is less than 20 wt-%, based on the total weight of the soft chewy formulation.

Therefore the present invention relates to a soft chewy formulation (SCF13′″), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″), (SCF9), (SCF10), (SCF10′), (SCF10″), (SCF11), (SCF12) or (SCF12), wherein the content of regular sugar is less than 15 wt-%, based on the total weight of the soft chewy formulation.

Therefore the present invention relates to a soft chewy formulation (SCF13′″″), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″), (SCF9), (SCF10), (SCF10′), (SCF10″), (SCF11), (SCF12) or (SCF12), wherein the content of regular sugar is 8 to 45 wt-%, based on the total weight of the soft chewy formulation.

Therefore the present invention relates to a soft chewy formulation (SCF13′″″), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″), (SCF9), (SCF10), (SCF10′), (SCF10″), (SCF11), (SCF12) or (SCF12), wherein the content of regular sugar is 8 to 35 wt-%, based on the total weight of the soft chewy formulation.

Therefore the present invention relates to a soft chewy formulation (SCF13′″″″), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″), (SCF9), (SCF10), (SCF10′), (SCF10″), (SCF11), (SCF12) or (SCF12), wherein the content of regular sugar is 10 to 30 wt-%, based on the total weight of the soft chewy formulation.

Therefore the present invention relates to a soft chewy formulation (SCF13′″″″), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″), (SCF9), (SCF10), (SCF10′), (SCF10″), (SCF11), (SCF12) or (SCF12), wherein the content of regular sugar is 8 to 15 wt-%, based on the total weight of the soft chewy formulation.

Furthermore, the soft chew according to the present invention can also comprise further active ingredients (next to the HMOs).

Preferred active ingredients for the embodiment of the present invention may include vitamins, nutritional lipids, minerals, nutraceuticals, glycosaminoglycan or its active members, amino acids or combination thereof that are useful in human or animal nutrition. preferably include vitamins, minerals, glycosaminoglycan or its active members and amino acids.

Such additional active ingredients may be present in amount of up to 35 wt-%, based on the total weight of the soft chewy formulation.

Therefore the present invention relates to a soft chewy formulation (SCF14), which is the soft chewy formulation (SCF), (SCF′), (SCF″), (SCF′″), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″), (SCF9), (SCF10), (SCF10′), (SCF10″), (SCF11), (SCF12), (SCF12), (SCF13), (SCF13′), (SCF13″), (SCF13′″″), (SCF13′″″), (SCF13′″″), (SCF13″″″), (SCF13′″″″) or (SCF13″″″″), wherein the soft chewy formulation comprise additional active ingredients chosen from the group consisting of vitamins, nutritional lipids, minerals, nutraceuticals, glycosaminoglycan or its active members and amino acids.

Therefore the present invention relates to a soft chewy formulation (SCF14′), which is the soft chewy formulation (SCF), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″), (SCF9), (SCF10), (SCF10′), (SCF10″), (SCF11), (SCF12), (SCF12), (SCF13), (SCF13′), (SCF13″), (SCF13′″), (SCF13″″), (SCF13′″″), (SCF13″″″), (SCF13′″″″) or (SCF13″″″″), wherein the soft chewy formulation comprise additional active ingredients chosen from the group consisting of vitamins, minerals, glycosaminoglycan or its active members and amino acids.

Such additional active ingredients may be present in amount of up to 35 wt-%, based on the total weight of the soft chewy formulation.

Therefore the present invention relates to a soft chewy formulation (SCF15), which is the soft chewy formulation (SCF), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″), (SCF9), (SCF10), (SCF10′), (SCF10″), (SCF11), (SCF12), (SCF12), (SCF13), (SCF13′), (SCF13″), (SCF13′″), (SCF13″″), (SCF13′″″), (SCF13″″″), (SCF13′″″″), (SCF13″″″″″), (SCF14) or (SCF14′), wherein the content of the additional active ingredients is up to 35 wt-%, based on the total weight of the soft chewy formulation.

It is clear, that all weight-percentages of the ingredients of one soft chewy formulation always add up to 100.

The soft chews of the invention may be packaged individually for administration and stable storage and then usually packed in a larger container. But it may also be possible not to pack them individually and then put them as such in a container.

A general way to produce the formulation according to the present invention is the following:

First prepare an oil mix including all oily components (such as the at least one oil) and the wetting agents by melting the oil and mixing with the wetting agents.

Afterwards mixing all dry ingredients in a mixer then add the prepared oil mix and adding al remaining ingredients such as humectants, wet binders and all other used ingredients while mixing at room temperature.

Knead the mix until uniform then form into soft chews to the desired weight and texture.

In a large scale, the soft chews according to the present invention are typically manufac-tured by blending and extrusion. Pre-mixed ingredients are introduced into an extruder barrel with a screw therein, then mixed, coagulated, expanded and sheared into a blended mixture, followed by application of additional heat if a harder texture is desired. Water introduced into the mixture must generally be of food or pharmaceutical grade, as it will be retained within the mixture. The blended mixture is then formed into a desired shape on a die plate, then cut into individual units.

Soft chews formulation comprises texturizer (such as tapioca syrup), fillers (such as tapioca starch), wetting agents (such as lecithin), sweeteners (such as Reb M), taste improv-ers (such as malic acid and flavors), binders (such as powder sugar), humectant (such as glycerin), oil moisturizer (such as coconut oil) and any other ingredients, which could be useful for the production of the core.

The shape and size of the soft chews according to the present invention can be any as commonly used. The shape can be square or rectangular or sphere-like. Or it can be in the any other form (animal, heart, or whatever wished).

The size should of the soft chews according to the present invention be such, that it can be consumed easily.

The soft chews according to the present invention can be taken as is or can be further coated by a usual coating.

In another embodiment of the present invention, the soft chew does not comprise any long chain polyunsaturated fatty acid (having more than 18 carbon atoms). This means that no long chain polyunsaturated fatty acid is added to the formulation intentionally. It may be possible that traces of long chain polyunsaturated fatty acids are present.

Therefore the present invention relates to a soft chewy formulation (SCF15), which is the soft chewy formulation (SCF), (SCF1), (SCF2), (SCF2′), (SCF3), (SCF3′), (SCF3″), (SCF4), (SCF4′), (SCF4″), (SCF5), (SCF5′), (SCF6), (SCF6′), (SCF6″), (SCF7), (SCF8), (SCF8′), (SCF8″), (SCF9), (SCF10), (SCF10′), (SCF10″), (SCF11), (SCF12), (SCF12), (SCF13), (SCF13′), (SCF13″), (SCF13′″), (SCF13″″), (SCF13′″″), (SCF13″″″), (SCF13′″″″), (SCF13″″″″″), (SCF14), (SCF14′) or (SCF15), wherein the soft chewy formulation does not comprise any long chain polyunsaturated fatty acid.

The following examples serve to illustrate the invention.

### EXAMPLES

All of the following examples have been made the following way:
1. Prepare fat mix by mixing coconut oil and lecithin, then place the fat mix in hot water bath at 50° C. Stir manually to keep it uniform.
2. Keep tapioca syrup in hot water bath at 50° C. Add color to the syrup and stir manually to keep it uniform.
3. Add the following ingredients into Hobart mixer: powder sugar, HMO, citric acid, tapioca starch, Reb M, solid flavors. Start mixing at speed setting 2, then gradually increase mixing speed to speed setting 4. Mix for 5 mins.
4. Reduce the mixing speed to speed setting 1, add fat mix (step 1) and wait for 1 minute, then add tapioca syrup (step 2), glycerin, and liquid flavor. Gradually increase mixing speed to speed setting 4. Mix for 5 mins.
5. Remove product from the mixer. Place it on a confectionery sheet then knead the product until it appears uniform.
6. Cut soft chews to the desired weight and wrap them.

### EXAMPLES

TABLE 1

| | Comparison Exp | Exp. 1 | Exp. 2 |
|---|---|---|---|
| Ingredient | [wt-%] | [wt-%} | [wt-%] |
| HMO 2′FL | — | 25.00 | — |
| HMO LNnT | — | — | 25.00 |
| Citric Acid | 0.20 | 0.17 | 0.25 |
| Powder Sugar | 41.70 | 26.46 | 30.83 |
| Tapioca Starch | 6.06 | 5.03 | 5.03 |
| Vegetable Glycerin | 0.90 | 0.75 | 0.75 |
| Organic Tapioca Syrup | 33.50 | 27.92 | 30.48 |
| Coconut Oil | 4.00 | 3.33 | 3.33 |
| Lecithin | 0.30 | 0.25 | 0.25 |
| Creamy Vanilla Flavor | 3.00 | 2.50 | 2.50 |
| Chocolate Type Flavor | 1.00 | 0.83 | — |
| Strawberry Cream Flavor | — | — | 1.00 |
| Reb-M | 0.12 | 0.08 | 0.08 |
| Cocoa Powder | 9.22 | 7.68 | |
| Strawberry Flavor | | | 0.50 |

TABLE 2

| | Exp. 3 | Exp. 4 |
|---|---|---|
| Ingredient | [wt-%] | [wt-%} |
| HMO 2′FL | 30.00 | |
| HMO LNnT | | 30.00 |
| Citric Acid | 0.20 | 0.20 |
| Powder Sugar | 11.70 | 11.70 |
| Tapioca Starch | 6.06 | 6.06 |
| Vegetable Glycerin | 0.90 | 0.90 |
| Organic Tapioca Syrup | 33.50 | 33.50 |
| Coconut Oil | 4.00 | 4.00 |
| Lecithin | 0.30 | 0.30 |
| Creamy Vanilla Flavor | 3.00 | 3.00 |
| Chocolate Type Flavor | 1.00 | 1.00 |
| Strawberry Cream Flavor | | |
| Reb-M | 0.12 | 0.12 |
| Cocoa Powder | 9.22 | 9.22 |
| Strawberry Flavor | | |
| Total | 100.00 | 100.00 |

As can be seen in Tables 1 and 2, five batches of soft chews have been produced using the same ingredients (except for flavors). A comparison example was produced as well, wherein the level of regular sugar (sucrose) is as high as 41.70%. Example 1 and example 2 used HMO 2′FL and HMO LNnT (at 25% w/w), respectively. It was successfully achieved to reduce the level of sucrose to 26.46% and 30.83%, respectively. For examples 3 and 4, the level of HMO 2′FL and HMO LNnT is 30% w/w in the formulation. For these two examples, it was achieved to reduce the sucrose to as low as 11.70%, (30% less than comparison example). In addition the texture and taste of all soft chew samples was also evaluated on the scale of 1-5. The results were summarized in Table 3. All samples have similar sensory ratings (based on average evaluations of 5 people).

TABLE 3

| | Comparison | | | | |
|---|---|---|---|---|---|
| | Exp. | Exp. 1 | Exp. 2 | Exp. 3 | Exp. 4 |
| Texture | 4 | 3.8 | 4.6 | 4.2 | 3.6 |
| Taste | 4 | 3.8 | 4.8 | 4.3 | 4 |

TABLE 4

| Rating | Description |
|---|---|
| 5 | Excellent |
| 4 | Good |
| 3 | Acceptable |
| 2 | Unacceptable |
| 1 | Really bad |

It can be seen, that the new soft chewy formulation according to the present invention are very similar to the ones known from the prior art and the reduction of the regular sugar is significant.

1. A soft chewy formulation comprising
(i) at least one humectant, and
(ii) at least one wetting agent, and
(iii) at least one oil, and
(iv) at least one binder, and
(v) at least 0.1-50 wt-%, based on the total weight of the soft chew formulation, of at least one HMO, and
and which has a water moisture content of less than 8 wt-%, based on the total weight of the soft chewy formulation.

2. The soft chewy formulation according to claim 1, having a hardness (measured by a textual analyser) in the range of 1.2 kg to 4.0 kg.

3. The soft chewy formulation according to claim 1, wherein the HMO are chosen from the group consisting of 2′-fucosyllactose (2′ FL), lacto-N-neotetraose (LNnT), 3-fucosyllactose (3FL), difucosyllactose (DFL), Lacto-N-fucopentaose I (LNFP I), 3′Sialyllactose Sodium Salt (3′SL), 6′Sialyllactose Sodium Salt (6′SL), and Lacto-N-Tetraose (LNT).

4. The soft chewy formulation according to claim 1, wherein the content of the at least one HMO is 5-45 wt-%, based on the total weight of the soft chewy formulation.

5. The soft chewy formulation according to claim 1, wherein the content of the at least one humectant is 0.5 to 8 wt-%, based on the total weight of the chewy formulation.

6. The soft chewy formulation according to claim 1, wherein the at least one humectant is a non-ionic polyol.

7. The soft chewy formulation according to claim 1, wherein the content of the at least one wetting agent is in the range of 0.05 to 4.0 wt-%, based on the total weight of the soft chewy formulation.

8. The soft chewy formulation according to claim 1, wherein the at least one wetting agent is chosen from the group consisting of lecithin (and lecithin derivatives), monoglyceride derivatives (such as ethoxylated monoglycerides, acetoglycerides and diacetyl tartaric esters of monoglycerides), fatty acid derivatives (such as polyglycerol esters (PGE), propylene glycol esters (PGMS), stearoyl lactylates, sucrose esters, sorbitan esters, polysorbates and sucrose esters), polysorbate 80 (which is a derivative of polyethoxylated sorbitan and oleic acid), and sorbitan monostearate.

9. The soft chewy formulation according to claim 1, wherein the content of the at least one oil is 1 to 10 wt-%, based on the total weight of the chewy formulation.

10. The soft chewy formulation according to claim 1, wherein the at least one oil is chosen from the group consisting of canola oil, soybean oil, sunflower oil, coconut oil, hemp oil, sunflower oil, soybean oil, pumpkin oil, cottonseed oil, corn oil, palm oil, castor bean oil, sesame oil, peanut oil, rapeseed oil and safflower oil.

11. The soft chewy formulation according to claim 1, wherein the content of the at least one binder is 10 to 45 wt-%, based on the total weight of the chewy formulation.

12. The soft chewy formulation according to claim 1, wherein the at least one binder is chosen from the group consisting of sucrose, sorbitol, mannitol, xylitol, liquid glucose, acacia, tragacanth, gelatin, starch, pregelatinized starch, alginic acid, cellulose, methyl cellulose, ethyl cellulose, hydroxy propyl methyl cellulose (HPMC), hydroxy propyl cellulose, sodium carboxy methyl cellulose, polyvinyl pyrrolidone (PVP), polyethylene glycol (PEG), polyvinyl alcohols and polymethacrylates.

13. The soft chewy formulation according to claim 1, wherein the soft chews comprise further ingredients chosen from the group consisting of bulking agents, sweeteners, nutritional lipids, flavoring agents, disintegrating agents, preservatives, lubricating agents, and anti-sticking agents.

14. The soft chewy formulation according to claim 1, wherein the soft chewy formulation comprises additional active ingredients chosen from the group consisting of vitamins, nutritional lipids, minerals, nutraceuticals, glycosaminoglycan or its active members and amino acids.
